(19) United States
(12) Patent Application Publication
(10) Pub. No.: US 2003/0229329 A1
(43) Pub. Date: Dec. 11, 2003

(54) Moisture absorbing garment system

(76) Inventors: Thomas G. Mercier, Houghton, MI (US); Traci L. Sleeman, Houghton, MI (US)

Correspondence Address: Michael S. Neustel, Suite No. 4, 2534 South University Drive, Fargo, ND, 58103, US

(21) Appl. No.: 10/162,954

(22) Filed: Jun. 6, 2002

(51) Int. Cl. A61F 13/15

(52) U.S. Cl. 604/394

(57) ABSTRACT

A moisture absorbing garment system for increasing the health, safety and confidence of a wearer utilizing a moisture absorbing garment. The moisture absorbing garment system includes a garment member having a front portion and a rear portion with side cutouts between the front portion and the rear portion for receiving the legs of a user, a piece of absorbing material within an inner portion of the garment member, a pair of front members extending from the front portion, and a plurality of rear members having fasteners for securing to the front members snugly and sealably about the waist and legs of the user.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND OF THE INVENTION

1. Field of the Invention

The present invention relates generally to moisture absorbing garments and more specifically it relates to a moisture absorbing garment system for increasing the total absorbency capacity and leak resistance thereby improving the health, safety and confidence of a wearer.

2. Description of the Related Art

Moisture absorbing garments have been in use for years for infants, children and adults with bodily waste evacuation challenges. Typically, conventional moisture absorbing garments are comprised of a shorts structure having a waist opening, a pair of leg openings for receiving the legs of an individual, and moisture absorbing material within the groin area.

The main problem with conventional moisture absorbing garments is that they do not provide a system for absorbing within the leg and inner thigh areas. Another problem with conventional moisture absorbing garments is that they typically do not have absorbing material in high-risk areas. A further problem with conventional moisture absorbing garments is that they do not provide absorption within the legs and inner thigh areas. Another problem with conventional moisture absorbing garments is that they do not provide protection for overnight or multi-movement wearing. A further problem with conventional moisture absorbing garments is that they do not provide adequate sealing at the inner thigh and leg crease and leg end areas.

Examples of patented devices which are related to the present invention include U.S. Pat. No. 6,293,937 to Matsushita et al.; U.S. Pat. No. 5,669,902 to Sivilich; U.S. Pat. No. 5,531,732 to Wood; U.S. Pat. No. 5,435,014 to Moretz et al.; U.S. Pat. No. 4,938,753 to Van Gompel et al.; U.S. Pat. No. 4,743,239 to Cole; U.S. Pat. No. 4,610,681 to Strohbeen et al.; and U.S. Pat. No. 3,613,687 to Kennedy.

While these devices may be suitable for the particular purpose to which they address, they are not as suitable for increasing the health, safety and confidence of a wearer utilizing a moisture absorbing garment. Conventional absorbing garments do not provide protection in the high-risk, inner thigh crease or leg end areas, or during overnight or multi-movement wearing.

In these respects, the moisture absorbing garment system according to the present invention substantially departs from the conventional concepts and designs of the prior art, and in so doing provides an apparatus primarily developed for the purpose of increasing the health, safety and confidence of a wearer utilizing a moisture absorbing garment.

### BRIEF SUMMARY OF THE INVENTION

In view of the foregoing disadvantages inherent in the known types of moisture absorbing clothing devices now present in the prior art, the present invention provides a new moisture absorbing garment system construction wherein the same can be utilized for increasing the health, safety and confidence of a wearer utilizing a moisture absorbing garment.

The general purpose of the present invention, which will be described subsequently in greater detail, is to provide a new moisture absorbing garment system that has many of the advantages of the moisture absorbing garments mentioned heretofore and many novel features that result in a new moisture absorbing garment system which is not anticipated, rendered obvious, suggested, or even implied by any of the prior art moisture absorbent garments, either alone or in any combination thereof.

To attain this, the present invention generally comprises a garment member having a front portion and a rear portion with side cutouts between the front portion and the rear portion for receiving the legs of a user, a piece of absorbing material within an inner portion of the garment member, a pair of front members extending from the front portion, and a plurality of rear members having fasteners for securing to and sealing the front members snugly and sealably about the waist and legs of the user.

There has thus been outlined, rather broadly, the more important features of the invention in order that the detailed description thereof may be better understood, and in order that the present contribution to the art may be better appreciated. There are additional features of the invention that will be described hereinafter and that will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

A primary object of the present invention is to provide a moisture absorbing garment system that will overcome the shortcomings of the prior art devices.

A second object is to provide a moisture absorbing garment system for increasing the health, safety and confidence of a wearer utilizing a moisture absorbing garment.

Another object is to provide a moisture absorbing garment system that increases the total absorbing material and fluid carrying capacity.

An additional object is to provide a moisture absorbing garment system that strategically places absorbing material within high-risk areas including those susceptible to multi-movement, nighttime or other positional leakage.

A further object is to provide a moisture absorbing garment system that utilizes a longer leg section.

Another object is to provide a moisture absorbing garment system that utilizes broad sealing members about the leg end for preventing flow around.

A further object is to provide a moisture absorbing garment system that fits various sizes of individuals.

Another object is to provide a moisture absorbing garment system for adults, children and infants.

Other objects and advantages of the present invention will become obvious to the reader and it is intended that these objects and advantages are within the scope of the present invention.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is an upper perspective view of the present invention.

FIG. 2 is a side view of the present invention closed.

FIG. 3 is a side view of the present invention open.

FIG. 4 is a cross sectional view taken along line 4-4 of FIG. 1.

### DETAILED DESCRIPTION OF THE INVENTION

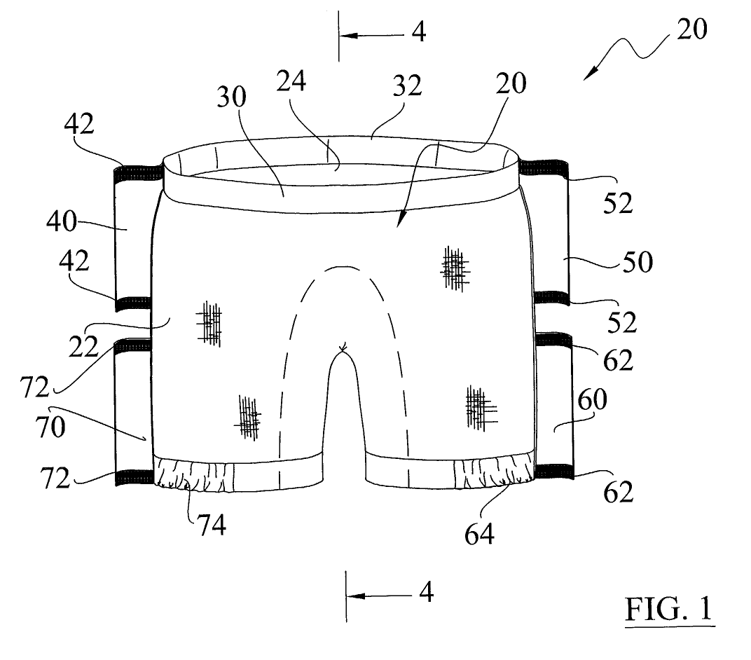

Turning now descriptively to the drawings, in which similar reference characters denote similar elements throughout the several views, FIGS. 1 through 4 illustrate a moisture absorbing garment system 10, which comprises a garment member 20 having a front portion 22 and a rear portion 24 with side cutouts between the front portion 22 and the rear portion 24 for receiving the legs of a user, a piece of absorbing material 80 within an inner portion of the garment member 20, a pair of front members 90 extending from the front portion 22, and a plurality of rear and leg end members having fasteners for securing to the front members 90 snugly and sealably about the waist and legs of the user.

FIGS. 1 through 4 illustrate the garment member 20 which has a generally broad structure for encompassing the lower torso and upper leg portions of a user when secured about the user. As shown in FIGS. 1 through 4 of the drawings, the garment member 20 includes a front portion 22 and a rear portion 24 connected to one another. The garment member 20 has a pair of opposing extended portions that extend downwardly for surrounding the upper leg portions of the user. The garment member 20 is preferably comprised of a fluid absorbent material for absorbing bodily fluids. The garment member 20 may be comprised of one or more layers of material capable of absorbing fluid. In addition, the garment member 20 may have an outer layer that is non-permeable for fluids thereby preventing the escape of fluids from the interior portion of the garment member 20 such as but not limited to polyethylene or other plastic.

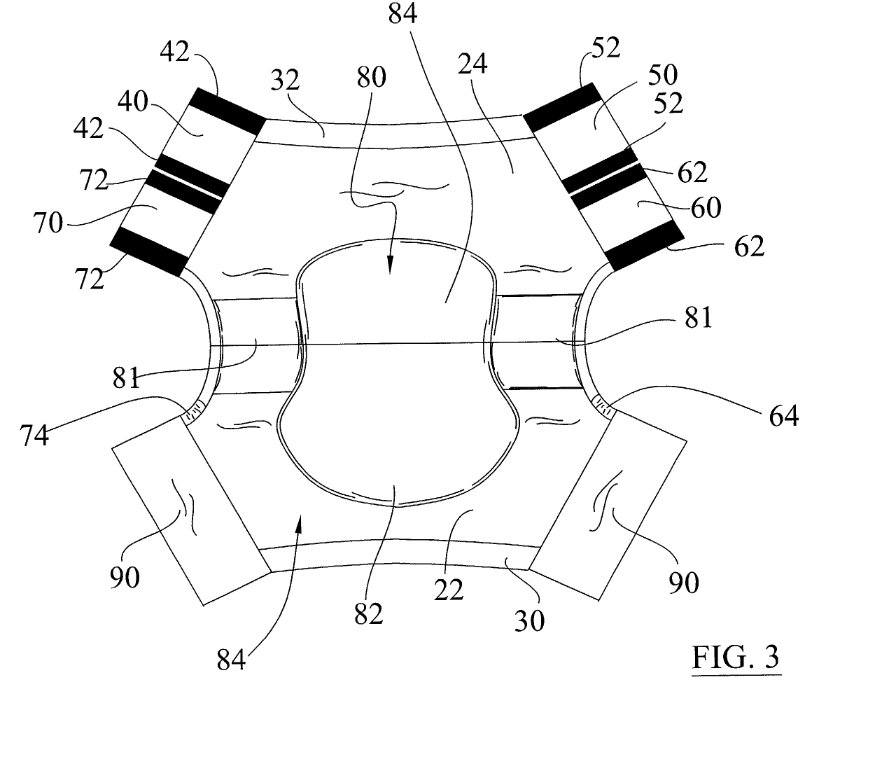

As shown in FIGS. 1 and 3 of the drawings, a front upper strap 30 is attached to the upper portion of the front portion 22. The front upper strap 30 is preferably a broader structure thereby forming a seal adjacent the user's skin during usage. The front upper strap 30 may be stretchable or non-stretchable.

As shown in FIGS. 1 and 3 of the drawings, a rear upper strap 32 is attached to the upper portion of the rear portion 24. The rear upper strap 32 is preferably a broader structure thereby forming a seal adjacent the user's skin during usage. The rear upper strap 32 may be stretchable or non-stretchable.

As shown in FIGS. 1 and 3 of the drawings, a first elastic segment 64 and a second elastic segment 74 are positioned within the lower edge of the front portion 22 of the garment member 20. The elastic segments 64, 74 allow for providing a tight fit adjacent the upper legs of the user thereby reducing the chance of leakage about and through the leg end. The extra length of the leg portion of the garment member 20 containing the side sections 81 acting as thigh absorbers cinched tightly to the side by fasteners and elastic members 64, 74 further reduces the likelihood of leakage around and through the inner thigh areas

As shown in FIGS. 1 and 3 of the drawings, a pair of front members 90 extend from the front portion 22 of the garment member 20. The front members 90 have a broad structure that extends a substantial portion of the vertical length of the garment member 20 when positioned upon a user.

As shown in FIGS. 1 and 3 of the drawings, a first member 40, a second member 50, a third member 60 and a fourth member 70 are attached to the rear portion 24 of the garment member 20 in opposition to the front members 90. The first member 40 and fourth member 70 are preferably positioned adjacent to one another as shown in FIG. 3 of the drawings. The second member 50 and third member 60 preferably are positioned adjacent to one another opposite of the first member 40 and the fourth member 70. The members 40, 50, 60, 70 preferably are comprised of a flexible material such as but not limited to cloth or plastic. The members 40, 50, 60, 70 may also be combined into a solid structure similar to the front members 90.

The members 40, 50, 60, 70 each have at least one fastener 42, 52, 62, 72 attached to the inner surface of the respective members 40, 50, 60, 70 as shown in FIG. 3 of the drawings. The fasteners 42, 52, 62, 72 are preferably attached in pairs upon each of the members 40, 50, 60, 70 on the distal portions of the members 40, 50, 60, 70 as shown in FIG. 3 of the drawings. The fasteners 42, 52, 62, 72 may be comprised of various types of fastening devices such as but not limited to adhesive, or hook and loop fastening structures. If an adhesive is utilized for the fasteners 42, 52, 62, 72, the front members 90 are preferably comprised of a material for removably receiving the adhesive in a secure manner. If a hook and loop fastener is utilized for the fasteners 42, 52, 62, 72, the front members 90 each include a corresponding mating hook and loop fastener for removably receiving the adhesive in a secure manner.

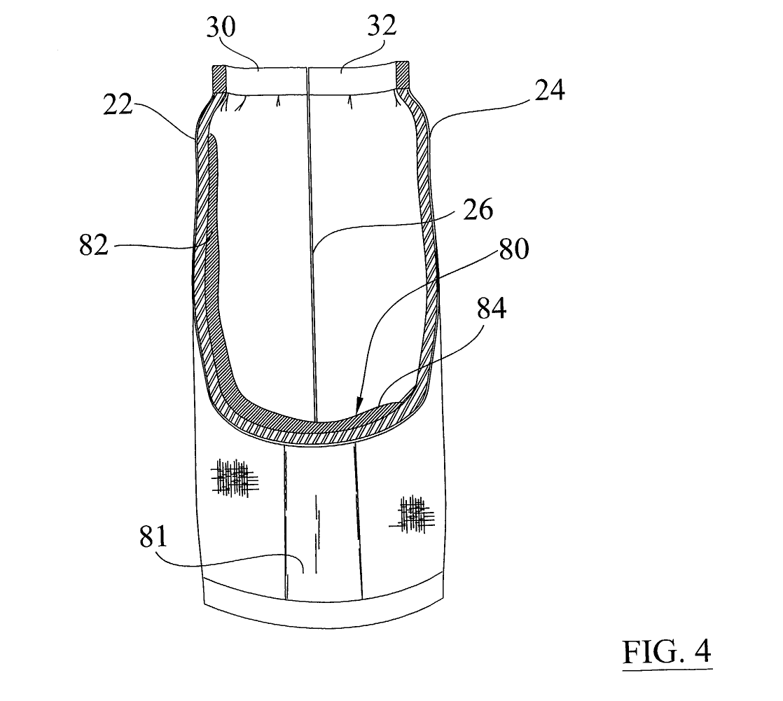

As shown in FIGS. 3 and 4 of the drawings, a piece of absorbing material 80 is positioned within an inner central portion of the garment member 20. The absorbing material 80 includes a front section 82, a rear section 84 and a pair of opposing side sections 81 as shown in FIG. 3 of the drawings. The absorbing material 80 is comprised of a highly absorbent material capable of receiving and storing a relatively significant amount of bodily fluid commonly utilized within the diaper industry such as but not limited to cellulose. Additional fluid absorbent materials may be attached to the absorbing material 80 such as but not limited to sodium polyacrylate.

The front section 82 of the garment member 20 preferably extends upwardly along the inner portion of the garment member 20 as best illustrated in FIG. 4 of the drawings. The rear section 84 preferably extends a finite distance rearwardly as shown in FIG. 4 of the drawings. The pair of opposing side sections 81 extend to the outer side portions of the garment member 20 for providing extra absorption near the leg/hip crease and inner thigh leg length areas when attached to the user.

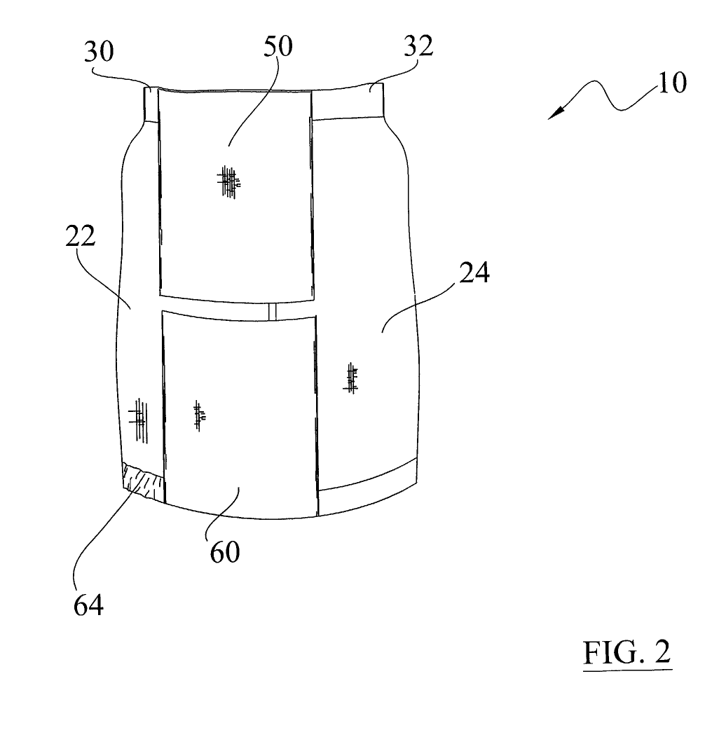

In use, the user positions the garment member 20 between the inner thighs and then pulls the front portion 22 and the rear portion 24 upwardly and together until snugly positioned about the waist and upper legs of the user. The user then secures the members 40, 50, 60, 70 to the front members 90 as shown in FIGS. 1 and 2 of the drawings. The wearer is then free to continue their normal activities without interference such as walking or sleeping. The absorbing material 80 absorbs and collects any urine or feces from the user thereby preventing the escape of the same from the garment member 20. If the user is lying within a bed, the rear portion 24 or the front portion 22 absorbs a significant portion of the urine fluid. The inner side sections 81 enhance fluid absorption thereby preventing flow around the leg/hip apex or down the inner thigh. In any position, the elastic members 64, 74 cinch tight and seal to the inner side sections 81. The elastic members 64, 74, also act as a seal preventing flow around or flow through the leg end. When finished using the present invention, the user simply removes the garment member 20 and disposes thereof.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed to be within the expertise of those skilled in the art, and all equivalent structural variations and relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

```
  10. Moisture Absorbing Garment System
  11.
  12.
  13.
  14.
  15.
  16.
  17.
  18.
  19.
  20. Garment Member
  21.
  22. Front Portion
  23.
  24. Rear Portion
□ 25.
□ 26.
□ 27.
□ 28.
□ 29.
□ 30. Front Upper Strap
□ 31.
□ 32. Rear Upper Strap
□ 33.
□ 34.
□ 35.
□ 36.
□ 37.
□ 38.
□ 39.
□
□
□
□
□
□
□
□
□ 40. First Member
□ 41.
□ 42. First Fastener
□ 43.
□ 44.
□ 45.
□ 46.
□ 47.
□ 48.
□ 49.
□ 50. Second Member
□ 51.
□ 52. Second Fastener
□ 53.
□ 54.
□ 55.
□ 56.
□ 57.
□ 58.
□ 59.
□ 60. Third Member
□ 61.
□ 62. Third Fastener
□ 63.
□ 64. First Elastic Segment
□ 65.
□ 66.
□ 67.
□ 68.
□ 69.
□ 70. Fourth Member
□ 71.
□ 72. Fourth Fastener
□ 73.
□ 74. Second Elastic Segment
□ 75.
□ 76.
□ 77.
□ 78.
□ 79.
□
□ 80. Absorbing Material
□ 81. Side Sections
□ 82. Front Section
□ 83.
□ 84. Rear Section
□
□ 90. Front Members
```

We claim:

1. A moisture absorbing garment system, comprising:
a garment member having a front portion and a rear portion, wherein when said garment member is positioned about a user said garment member extends a finite distance downwardly along the legs of a user;
a pair of front members extending from said front portion of said garment member;
a plurality of rear members extending from said rear portion of said garment member;
a fastener attached to each of said plurality of rear members for removably securing to said pair of front members; and
a piece of absorbing material attached to an inner portion of said garment member.

2. The moisture absorbing garment system of claim 1, wherein said garment member is comprised of an absorbent material.

3. The moisture absorbing garment system of claim 1, wherein said piece of absorbing material has a front section extending a finite distance upwardly along said front portion.

4. The moisture absorbing garment system of claim 3, wherein said piece of absorbing material has a rear section extending a finite distance upwardly along said rear portion.

5. The moisture absorbing garment system of claim 4, wherein said piece of absorbing material has a pair of side sections each extending a finite distance outwardly to engage the opposing inner thighs of a user.

6. The moisture absorbing garment system of claim 5, wherein said side sections each extend to a respective side edge of said garment member.

7. The moisture absorbing garment system of claim 1, wherein said piece of absorbing material has a pair of side sections each extending a finite distance outwardly to engage the opposing inner thighs of a user.

8. The moisture absorbing garment system of claim 7, wherein said side sections each extend to a respective side edge of said garment member.

9. The moisture absorbing garment system of claim 1, wherein said piece of absorbing material has a rear section extending a finite distance upwardly along said rear portion.

10. The moisture absorbing garment system of claim 1, wherein said plurality of rear members are comprised of two pairs attached to opposing sides of said rear portion.

11. A moisture absorbing garment system, comprising:
a garment member having a front portion and a rear portion, wherein when said garment member is positioned about a user said garment member extends a finite distance downwardly along the legs of a user;
a first elastic segment and a second elastic segment attached to said front portion in opposition to one another;
a pair of front members extending from said front portion of said garment member;
a plurality of rear members extending from said rear portion of said garment member;
a fastener attached to each of said plurality of rear members for removably securing to said pair of front members; and
a piece of absorbing material attached to an inner portion of said garment member.

12. The moisture absorbing garment system of claim 11, wherein said garment member is comprised of an absorbent material.

13. The moisture absorbing garment system of claim 11, wherein said piece of absorbing material has a front section extending a finite distance upwardly along said front portion.

14. The moisture absorbing garment system of claim 13, wherein said piece of absorbing material has a rear section extending a finite distance upwardly along said rear portion.

15. The moisture absorbing garment system of claim 14, wherein said piece of absorbing material has a pair of side sections each extending a finite distance outwardly to engage the opposing inner thighs of a user.

16. The moisture absorbing garment system of claim 15, wherein said side sections each extend to at least a respective side edge of said garment member.

17. The moisture absorbing garment system of claim 11, wherein said piece of absorbing material has a pair of side sections each extending a finite distance outwardly to engage the opposing inner thighs of a user.

18. The moisture absorbing garment system of claim 17, wherein said side sections each extend to a respective side edge of said garment member.

19. The moisture absorbing garment system of claim 11, wherein said piece of absorbing material has a rear section extending a finite distance upwardly along said rear portion.

20. The moisture absorbing garment system of claim 11, wherein said plurality of rear members are comprised of two pairs attached to opposing sides of said rear portion.
